(19) United States
(12) Patent Application Publication
(10) Pub. No.: US 2021/0371786 A1
(43) Pub. Date: Dec. 2, 2021

(54) MICROFLUIDIC DEVICES FOR DENTAL APPLICATIONS

(71) Applicant: OREGON HEALTH & SCIENCE UNIVERSITY, Portland, OR (US)

(72) Inventor: Luiz E. Bertassoni, Portland, OR (US)

(21) Appl. No.: 17/282,828

(22) PCT Filed: Oct. 3, 2019
(86) PCT No.: PCT/US2019/054578

§ 371 Date: Apr. 5, 2021

Related U.S. Application Data
(60) Provisional application No. 62/740,926, filed on Oct. 3, 2018.

(51) Int. Cl. C12M 3/06; A61B 10/00; C12M 1/00; C12M 3/00; C12M 1/12

(52) U.S. Cl. CPC C12M 23/16; A61B 10/0096; C12M 25/14; C12M 21/08; C12M 29/10

(57) ABSTRACT

Disclosed is a microfluidic device having a channel a chamber in fluid communication with the channel. The chamber is sized to contain dental sample that is form a semipermeable barrier through which fluid from the channel is deliverable to the dental sample.

### RELATED APPLICATION

This application claims priority benefit of U.S. Provisional Patent Application No. 62/740,926, filed Oct. 3, 2018.

### FEDERALLY SPONSORED RESEARCH OR DEVELOPMENT

This invention was made with government support under R01DE026170 awarded by the National Institutes of Health (NIH). The government has certain rights in the invention.

### TECHNICAL FIELD

This disclosure relates generally to microfluidic devices and, more particularly, to tooth-on-a-chip devices.

### BACKGROUND INFORMATION

Previous attempts to study biological samples have included so-called organ-on-a-chip systems. Organ-on-a-chip systems represent an emerging field that integrates micro-engineered substrates, microfluidics, tissue engineering, and biosensing for replicating tissue functionality that is challenging to replicate using conventional two- or three-dimensional cell culture systems. Thus, miniaturized organ-on-a-chip systems facilitate experimental control over multifactorial questions that are challenging to systematically study in-vivo.

Organ-on-a-chip technologies are employed in addressing biological questions of complex systems that are naturally responsive to flow, loading, and heterotypic cell contact. Several organ-on-a-chip models have demonstrated abilities to replicate complex multicellular architecture, cell-cell and cell-matrix interactions, physicochemical gradients, and fluid-flow conditions or tissue mechanics that are present naturally in complex tissues. For instance, a gut-on-chip-model attempted to replicate the human gut epithelial microvilli, vasculature, microflora, and peristalsis. Other attempts include liver-on-a-chip, lung-on-a-chip, bone-marrow-on-a-chip, and several others.

### SUMMARY OF THE DISCLOSURE

Disclosed is a microfluidic device comprising a channel and a chamber in fluid communication with the channel and sized to contain a dental sample, the dental sample configured form a semipermeable barrier through which material from the channel is deliverable to the dental sample. Moreover, disclosed tooth-on-a-chip model systems (or simply, tooth-on-a-chip or pulp-dentin on-a-chip) comprises mold-formed polydimethylsiloxane (PDMS) having ports (e.g., reservoirs or inlets and outlets for perfusion) and associated fluid channels in fluid communication with a dental sample (e.g., a dentin fragment). The PDMS includes mounting surfaces to which the dental sample is retained inside the chip.

The disclosed devices may be used in different forms, based on a combination of material or cells placed on the dentin surfaces. Several uses are listed as follows: the device can be used to (i) investigate the effect of different dental treatment as biomaterials, light-based treatments or medication on dental pulp cells morphology, gene expression and secretome by applying the treatment on one side of the dentin surface and culturing the pulp cells on the other; (ii) investigate interactions of dental pulp cells with either single microorganisms strains or patient's microbiome to obtain information on gene, protein, cellular and tissue levels; (iii) analyze the vasculature, innervation, and immune cells of the dental pulp by culturing cells embedded in a extracellular matrix interfacing with a dentin surface; (vi) investigate the interaction between saliva/microorganisms and enamel-dentin-pulp cells, e.g., by placing fragments of enamel and dentin in a central groove of the device, having saliva with or without microorganisms flowing through one channel, and dental pulp cells cultured on the opposite side of the dentin; (v) emulate enamel and dentin caries formation and to test remineralization strategies; (vi) investigate dental pulp stem cell niches; and (vii) test the effect of masticatory movements on dental pulp cells biology, e.g., by introducing mechanical deformation of the PDMS using of vacuum chambers fabricated on the sides of the device and connected to pumps. These aforementioned applications can be performed using dentin from either permanent or deciduous teeth, thereby expanding the applicability of the device to study of biomaterials, microbiology, immune responses and pulp regeneration to both primary and permanent dentition.

More specifically, the disclosed tooth-on-a-chip has applicability in the field of dental regeneration and may be used to analyze biocompatibility and cytotoxicity of dental materials or, for example, study the influence of the dental pulp innervation on odontogenesis during pulp regeneration or investigating gelatinolytic activity while simultaneously imaging a hybrid layer as formed by adhesive systems.

A tooth-on-a-chip also allows for integrating cells cultured directly on a dentin wall within a microdevice that replicates the architecture and dynamics of the dentin-pulp interface. This enables an improved understanding of the morphologic, metabolic, and functional influence of biomaterials on live dental pulp cells. For example, a tooth-on-a-chip enables real-time tracking of odontoblast monolayer formation, remodeling, and death in response to biomaterial treatments. Accordingly, the tooth-on-a-chip provides a platform for replicating near-physiologic conditions of the pulp-denting-biomaterials interface and enables live-cell imaging to study dental pulp cell response to biomaterials. The tooth-on-a-chip provides direct visualization of the complexity of the pulp-denting-biomaterials interface and enables real-time assessment of the response of pulp cells to dental materials.

Other example experiments facilitated by the disclosed devices include determining the role of soluble neurotrophic factors transiently immobilized in the native dentin on the process of neuronal repair; systematic study of mutual trophic effect of dental pulp innervation and mineralized dentin formation and characterizing axonal growth on the de-novo dentin formation at the pulp-dentin interface; and analyzing the influence of peripheral neurons on new dentin formation. It is also possible to determine conditions that enhance engineering of the dental pulp innervation both in-vitro and in-vivo. To facilitate dental research, the disclosed tooth-on-a-chip provides in-vitro models replicating multi-tissue interactions occurring in the pulp, such as its constant fluid-flow, hydrostatic pressure, organized 3D architectures, as well as its spatiotemporal chemical gradients.

Additional aspects and advantages will be apparent from the following detailed description of embodiments, which proceeds with reference to the accompanying drawings.

### BRIEF DESCRIPTION OF THE DRAWINGS

FIGS. 1A, 1B, 1C, 1D, 1E, and 1F are a sequence of perspective views showing fabrication techniques for tooth-on-a-chip devices having reservoirs (FIGS. 1A-1D, according to one embodiment) and perfusion inlets and outlets (FIGS. 1E and 1F, according to another embodiment).

FIGS. 2A and 2B are top plan views showing a microfluidic device, respectively, without and with the placement of a dental sample in a central chamber, in which FIG. 2B also shows an optional cell-laden matrix in a first side of the central chamber adjacent a first side of the dental sample.

FIGS. 3A and 3B are annotated pictorial views showing a variant of the embodiments of FIGS. 2A and 2B in which a central channel retains the cell-laden matrix shown in FIG. 2B.

FIGS. 4A and 4B are top plan views of other embodiments showing central chambers including microfabricated grooves through which axonal processes extend towards a hydrogel central chamber, according to some experiments.

FIGS. 4C, 4D, 4E, and 4F are microscopic pictorial views showing in greater detail the following four items shown in FIG. 4B: dentin, a hydrogel microenvironment encapsulated with stem cells from the apical papilla (SCAP), microfabricated grooves, and neurites.

### DETAILED DESCRIPTION OF EMBODIMENTS

Treatment of some dental diseases, such as caries or dentin hypersensitivity, entails application of a biomaterial directly onto a cavity formed on the tooth surface. Thus, these procedures typically involve attachment of the biomaterial onto dentin, i.e., the calcified tissue underlying the outer dental enamel. The structural configuration of the tooth itself, therefore, results in the formation of an interface in which biomaterials contact the dentin matrix, which thereby (indirectly) allows reaction byproducts and leachates to diffuse into the underlaying dental pulp. Such interactions are possible due to dentinal tubules that are about two micrometers in diameter and are distributed across the dentin matrix. The dentinal tubules house odontoblast cell processes that extend a significant length into the dentinal tubules. Therefore, together with the dental pulp, a restored tooth forms an intricate biomaterials/dentin/cell complex that is unique within the human body.

Biocompatibility and cytotoxicity of dental materials on pulp cells have been studied in the prior art. For instance, existing model systems include cells cultured on plates, larger devices employing specialized equipment such as in the Hume model, an in-vitro pulp chamber, a dentin barrier test, ex-vivo models such as the rodent slice culture, and entire human tooth cultures. These models, however, afford limited opportunities for direct observation (i.e. live cell imaging) of the morphologic and metabolic events that occur as cells inside of the tooth become exposed to biomaterials over time. It is the inventor's present belief that the aforementioned morphologic and metabolic events provide useful information regarding the ability of the tooth to respond to different biomaterials and treatments.

The present inventor also recognized that direct observation of dental pulp innervation is desirable as it is a regulator of the dental homeostasis. In addition to their nociceptive signaling, neurons innervating the pulp secrete a multitude of factors that regulate blood flow, cell differentiation and function in a paracrine, autocrine and juxtacrine manner. Such neuron-derived factors have been shown to contribute to mineralized tissue formation, and successful pulp-dentin regeneration is facilitated by an ability to understand and engineer three-dimensional (3D) innervated tissues. Thus, biomaterials-driven strategies to understand, manipulate, and engineer the myriad aspects regulating the dental pulp innervation are desirable, especially in the context of regenerative endodontics. But direct observations of such phenomena have remained challenging.

This disclosure, therefore, describes micro- and nanoengineering techniques for development and use of a tooth-on-a-chip model (e.g., as a microphysiologic on-chip model of dental pulp regeneration). Also described are example embodiments for mimicking the process of material (e.g., drug, mRNA or other biologic, or other dental treatment material) delivery through the dentin tubules; nano-delivery-based strategies to control the secretome of dental stem cells for enhanced neuronal repair; and systematically determination of conditions that enhance engineering of the dental pulp innervation both in-vitro and in-vivo.

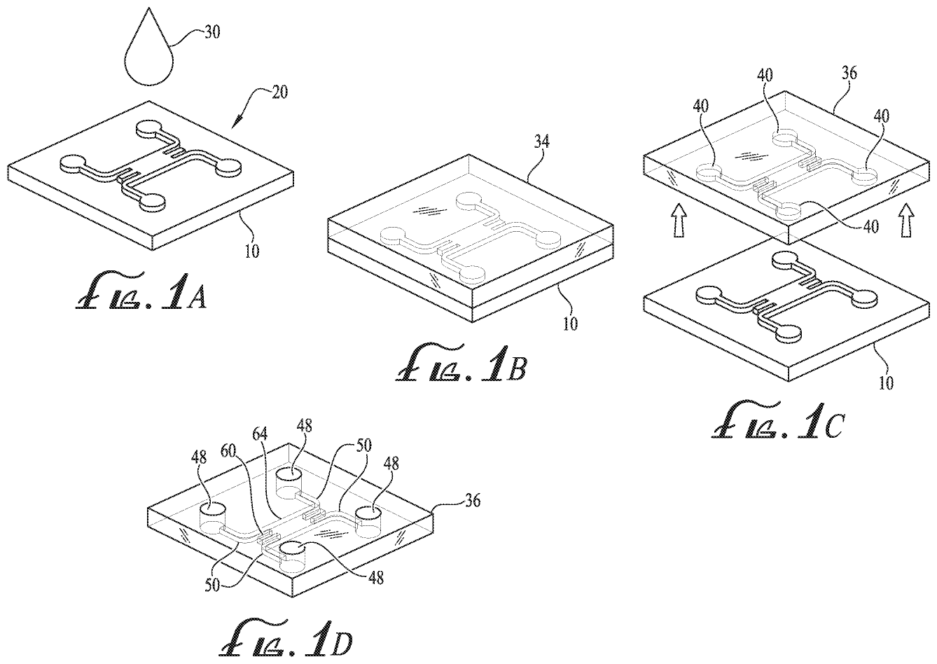

FIG. 1A shows a master, positive poly-methyl methacrylate (PMMA) mold 10 providing a positive template 20 for creating a tooth-on-a-chip 24 (see e.g., FIG. 1F). Template 20 is cut from a one mm-thick sheet of PMMA board using a Boss LS1416 laser cutter available from Boss Laser, LLC of Sanford, Fla. Templates are attached to the base of an impression container (not shown).

Polydimethylsiloxane (PDMS) prepolymer 30 is poured atop positive template 20 to form a transparent layer 34, as shown in FIG. 1B. Layer 34 is cured for about two hours (e.g., overnight) at 80° C. to form an oxygen-permeable, biocompatible polymer. Skilled persons will appreciate that other biologically inert materials may be used for the body of a chip, such as, for example other silicone polymeric materials. The term “inert” refers to a quality of a material to have little or no inherent power of action, motion, or resistance in regard to other materials, cells, or agents used in a design or device described herein. For instance, the term “biologically inert” refers to a substance or material that does not enhance or detract from the activity of biologically active materials, cells, or agents used in a design or device described herein.

Skilled persons will now also appreciate that the body portions of the present embodiments may be made of materials known for use in microfluidics devices, including metal materials; silica-based materials such as glass, quartz, silicon, fused silica, or the like. Useful polymeric materials (or a polymer coating on other materials) include acrylics (e.g., polymethylmethacrylate (PMMA)), polycarbonate, polytetrafluoroethylene (PTFE), fluorinated ethylene propylene (FEP), polyvinylchloride, polydimethylsiloxane (PDMS), polysulfone, polystyrene (PS), polymethylpentene, polypropylene, polyethylene, polyvinylidine fluoride, acrylonitrile-butadiene-styrene copolymer, cyclic olefin copolymer (COC), cyclic olefin polymers (COP), or the like; ceramic materials, etc. Also, depending upon the specific dimensions of the desired use, specific areas of a microfluidic device can be lined with different substances than the substance of which the rest of the microfluidic device is composed.

Silicone polymeric or elastomeric materials that may be used in the present embodiments include those formed from precursors including the chlorosilanes such as methylchlorosilanes, ethylchlorosilanes, and phenylchlorosilanes, and the like. One useful silicone elastomer is polydimethylsiloxane (PDMS). Exemplary polydimethylsiloxane polymers include those sold under the trademark Sylgard by Dow Chemical Co., Midland, Mich., and particularly Sylgard 182, Sylgard 184, and Sylgard 186.

Cured PDMS 36 (FIG. 1C) forms a biologically inert body in which fluid-flow features are negatively molded (i.e., recessed) once cured PDMS (or simply, PDMS) 36 is released from PMMA mold 10. For example, FIG. 1C shows PDMS 36 is fabricated with four reservoirs 40 (e.g., for retaining cell medium).

To fully form reservoirs 40, FIG. 1D shows that relatively small, e.g., eight-millimeter (mm), holes 48 are punched at ends of fluid channels 50. Thus, each reservoir 40 is about eight mm in diameter and is about 6 mm deep.

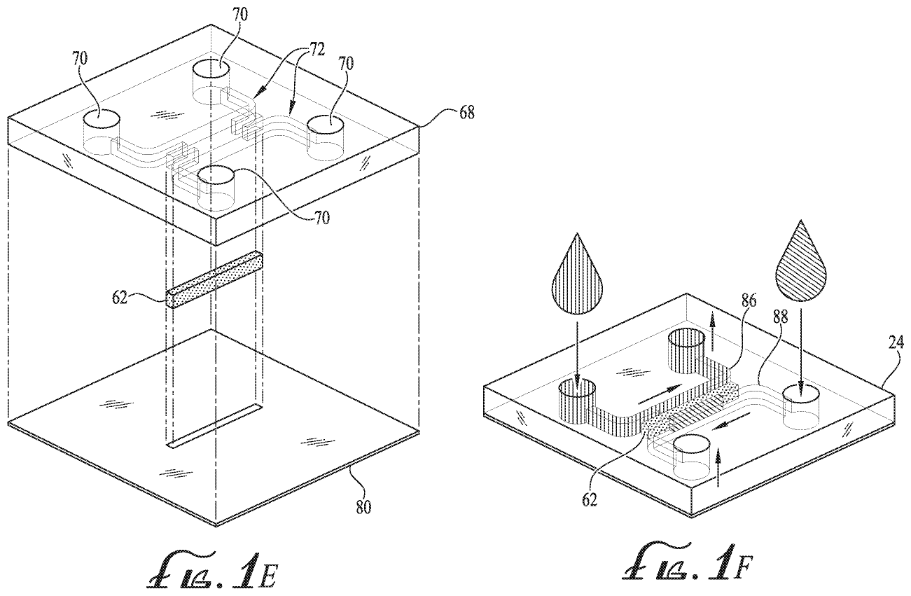

FIG. 1D also shows PDMS 36 is formed with a central groove 60 (see e.g., FIG. 2A) and associated ridges to hold a dental sample such as a dentin fragment 62 (FIG. 1E). Groove 60 widens along its mid-section to define a central chamber 64, with each side portion of chamber 64 being in fluid communication with a corresponding pair of static reservoirs 40 on the same side, thereby providing on each side of groove 60 a U-shaped channel that is about 1 mm wide and 0.10 mm tall. Each side portion of chamber 64 is about 300 micrometer (μm) in width (W)×one mm in length (L)×one mm in height (H) (see e.g., FIG. 2A), in the present example. Accordingly, as described later, cells may be placed in a side portion of chamber 64 with dentin fragment 62 acting as a semipermeable barrier that separates fluid channels 50, thereby defining microenvironments on each side of dentin fragment 62. Fluid (e.g., carrying dental materials) is deliverable through channels 50 and into chamber 64 using a variety of techniques such by differences in fluid depths or pressures of each reservoir 40. Static or no fluid flow is also possible, depending on the experimental set up. In other words, some embodiments may have a fluid-flow rate of 0 ml/h, and it may lack optional inlet-outlet ports.

The term “semipermeable barrier” refers to a material, membrane, or other barrier that allows certain substances to pass through, but not others. In some embodiments, the semipermeable barrier allows the passage of oxygen or other gases, nutrients, testing agents, etc. to pass into the desired portion of the device or design in question. Examples of semipermeable barriers include sheets or membranes comprising molydimethylsiloxane (PDMS), poly(ethylene glycol) diacrylate (PEGDA), chitosan, track-etched polymer membranes, and the like. In some embodiments, the semipermeable barrier is a membrane or sheet of material having pore openings of a desired size. In some embodiments, the pore size is at least 0.1 nm. In other embodiments, the pore size if from about 10 μm to about 100 μm.

Sizes and configuration of holes 48, channels 50, and chamber 64 may vary depending on the application. For instance, FIGS. 1E and 1F show another embodiment of a PDMS 68 having slightly smaller holes 70 intended for use as inlets and outlets for two perfusable channels 72. Still other embodiments may include two or more reservoirs (e.g., one reservoir on each side of a dental sample), one or more inlets, one or more outlets, and various combinations thereof. Accordingly, the terms reservoirs, inlets, and outlets are more generally referred to as fluid or material ports, or simply, ports. For example, the term “perfusion port” refers to an opening, passageway, portal, aperture, or valve structure for fluid communication or the intake or exhaust of perfusing, diffusing, or suffusing matter in relation to devices and designs described herein.

Before assembling a tooth-on-a-chip device, a tooth fragment is cut using a low speed saw (or similar device). In the present example, dentin fragment 62 is cut to 500 μm W×one mm H×4.5 mm L, and it is cut perpendicular to the dentin tubules. Detin is available as human dentin from third molars extracted for orthodontic reasons.

PDMS 68 and a glass coverslip 80 (FIGS. 1E and 1F) are cleaned using a plasma cleaner model PDC-32G available from Harrick Plasma of Ithaca, N.Y. PDMS plasma treatment increases silanol groups (—OH) at the surface of PDMS 68 so it forms strong covalent bonds (Si—O—Si) when brought together with glass coverslip 80. Dentin fragment 62 is not submitted to plasma treatment to avoid a chemical change in its structure.

After plasma treating PDMS 68 and coverslip 80, dentin fragment 62 is carefully inserted into groove 60 (e.g., using tweezers). Thus, dentin fragment 62 is manually inserted (pressure fit) to split pre-fabricated chamber 64.

Once dentin fragment 62 is inside groove 60, PDMS 68 is positioned against glass coverslip 80 and glass coverslip 80 is pressed (e.g., using clamps) onto a confronting surface of PDMS 68 using slight pressure, thereby forming a sealed and leak-proof microdevice, i.e., tooth-on-a-chip 24 (FIG. 1F).

FIG. 1F also shows chip 24 in operation such that dentin fragment 62 establishes a semipermeable membrane between divided portions of chamber 64, thereby creating distinct microenvironments for each side of chamber 64. For example, a first chamber 86 and a second chamber 88 represent, respectively, a pulp side and a cavity side. Thus, the fully assembled microdevice may replicate on one side an interface between dental pulp and dentin and—on the other side—an interface between dental (treatment) materials and dentin, thus forming two accessible chambers representing the pulp side and the cavity side, respectively.

Material passing through channels in the embodiments herein may include biologically acceptable media. The term “media” herein refers to is any fluid, such as conventional cell culture medias, that contain nutrition, oxygen or other gases, or other elements needed for the maintenance, growth, or testing of cells or agents in one or more of the designs and devices herein. In some embodiments, the media comprises a fluid capable of delivering other biological molecules to the domains, including, but not limited to, growth factors, chemokines, cytokines, hormones, etc. In some embodiments, the media comprises one or more drugs or other biologically or medically active compounds to be tested for effects in bone marrow and/or the cells of the specific design or device.

FIG. 1F also shows that, although dentin fragment 62 is about five mm in length, optional borders placed within groove 60 of PDMS 68 expose to perfusion a portion of the surface area of fragment 62. For instance, hatched-line regions show an exposed portion that is about 2-2.5 mm L×one mm H×0.5 mm W. Additional examples of optional borders are shown in FIGS. 2A and 2B.

In some embodiments, microfluidic devices are sterilized with ultraviolet light for about 40 minutes before use. An example light source is an EXFO Acticure 4000, which produces 365 nm light, applied at 8.5 cm distance with a light density of 45 milliwatts per square cementer (mW/cm²). To inhibit dentin dehydration after sterilization, a tooth on-a-chip is then filled with sterile water until use.

FIGS. 2A and 2B show another microdevice 90 including two opposing V-shaped perfusion channels. A first contained channel 100 and a second contained channel 102 are disposed on opposite sides of a central chamber 108, which FIG. 2B shows divided by a dental sample, such as a dentin sample 120 maintained in central chamber 108. As described previously, placement of dentin sample 120 creates a sealed arrangement separating first contained channel 100 from second contained channel 102 (i.e., thereby dividing central chamber 108). In this arrangement, a first fluid flow may be maintained in first contained channel 100 between inlet 130 and outlet 136. A second fluid flow may be maintained in second contained channel 102 between inlet 140 and outlet 144. Dashed arrows in the represent a flow of dental materials therethrough, although skilled persons will appreciate that other flow directions are possible.

FIG. 2B also shows an example in which a microfluidic device includes a pulp-like tissue construct interfaced with native dentin, and which may be cultured under controlled fluid-flow. Specifically, a cell-laden matrix 150, such as a cell-laden hydrogel (explained in the following paragraph), may be maintained in one contained channel adjacent dentin sample 120. Chamber 108, therefore, is sized to accommodate cell-laden matrix 150.

The term “hydrogel” as used herein refers to a gel comprising a cross-linked network of water-soluble polymers capable of forming a matrix mimicking a natural extracellular matrix and supporting the biological materials and activities of interest to the present studies. Commercially available hydrogels include the MATRIGELTM matrix (available from Corning Inc., Tewksbury, Mass.); poly[2-(methacryloyloxy)ethyl dimethyl(3-sulfopropyl)ammonium] (PMEDSAH) hydrogels or copolymers or blends thereof; glycoprotein hydrogels, such as fibronectin hydrogels and laminin hydrogels; protein hydrogels, such as those derived from collagen, albumin, fibrin, or silk proteins; polysaccharide hydrogels, such as those derived from glucan, hyaluronic acid, chitosan, agarose, and alginate; synthetic hydrogels composed of synthetic monomers such as those selected from the group of poly(ethylene glycol) (PEG), poly(vinyl alcohol) (PVA), poly(ethylene oxide) (PEO), poly(acrylic acid) (PAA), poly(hydroxyethyl methacrylate) (PHEMA), poly(methacrylic acid) (PMMA), polypropylene fumarate-co-ethylene glycol (P(PF-cop-EG)), poly(acrylamide) (PAAm), and poly N-isopropylacrylamide (PNIPAAm); and hybrid synthetic-biologic hydrogels having combined monomers of synthetic and biological materials, such as PEG-peptide hydrogels, including PEG-fibrinogen hydrogels. The term “hydrogel”, as used herein, is understood to include a single type of hydrogel material, such as one of the individually listed hydrogel materials above, or a mixture or combination of two or more individual hydrogel materials, such as a combination of the MATRIGELTM matrix with collagen and/or a fibronectin hydrogel.

In some embodiments, the matrix comprises collagen fibers. In some embodiments, the collagen fibers are selected from the group of collagen type I (also known as Type 1 collagen), collagen type II, collagen type III, collagen type IV, collagen type V, collagen type VI, collagen type VI, collagen type VII, collagen type VIII, collagen type IX, collagen type X, collagen type XI, collagen type XII, collagen type XIII, collagen type XIV, collagen type XV, collagen type XVI, collagen type XVII, collagen type XVIII, collagen type XIX, and collagen type XX, or a combination thereof. In some embodiments the hydrogel matrix comprises type I collagen.

“Type I collagen” or “Type 1 collagen” refers to the fibrillar-type collagen that is the most abundant form of human collagen and the key structural composition of several tissues.

The embodiment of FIG. 2B may be used to observe in real-time how pulp cells respond to various dental materials used clinically (e.g., phosphoric acid, adhesive monomers, and dental adhesives) as these materials flow through the cavity side. For example, the materials may be tested for cytotoxicity, cell morphology, and metabolic activity on-chip, and compared against standardized off-chip controls. To characterize pulp cell responses to dental materials on-chip, stem cell-derived odontoblasts may be seeded onto the dentin surface and observed using live-cell microscopy. For instance, 2-hydroxyethylmethacrylate (HEMA), Phosphoric Acid (PA), and Adper-Scotchbond (SB) are testable so that comparisons can be made between an engineered pulp tissue interfaced with the native human dentin in and an ISO in-vitro model. Additional experiments are the subject of a paper titled, “The Tooth on-a-chip: a Microphysiologic Model System Mimicking the Pulp-Dentin Interface and its Interaction with Biomaterials,” by the present inventor and other co-authors, and made available as of Aug. 28, 2019 from bioRxiv.org.

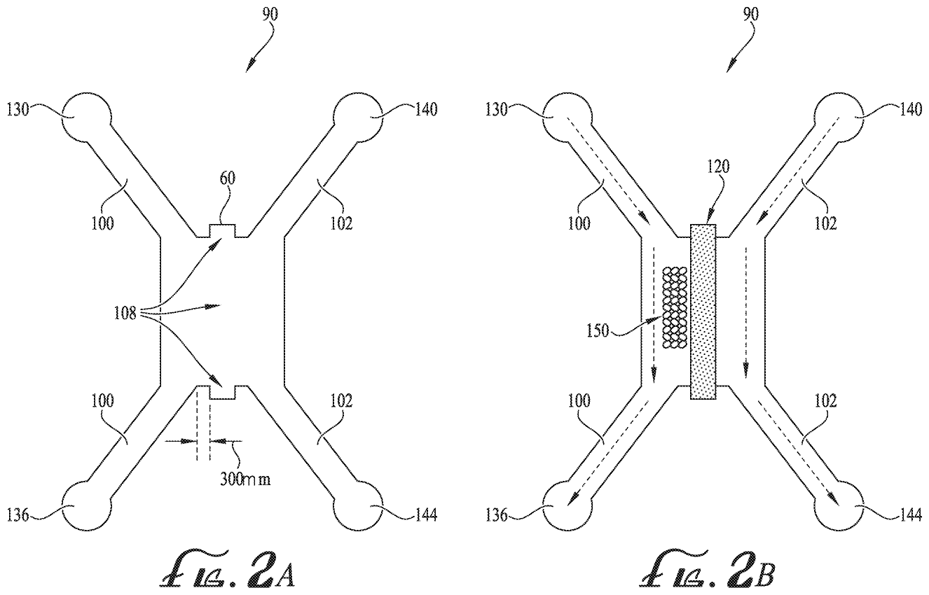

FIGS. 3A and 3B show another a pulp-dentin microdevice 300. Initially, for completeness, it is noted that microdevice 300 is fabricated as described previously, measuring 200 μm H×1.5 mm W×seven mm L. For instance, FIG. 3A shows that, after PDMS 306 sets, molded features form negative channels that are covalently bonded to a glass slide 308 using a plasma etcher, thus forming a sealed and leak-free system.

Microdevice 300 is similar to that shown in FIG. 1E, but microdevice 300 also includes a central gel-loading channel 310 in fluid communication with a central chamber 314 in which a cell-laden collagen hydrogel functions as a pulp-like surrogate. The pulp-like surrogate is connected to an adjacent microchannel 316 enabling perfusion of medium to the cells.

A dentin fragment 318 has been precision-cut using an automated computer numerical control (CNC) dental milling machine to the desired thickness (perpendicular to the dentin tubules), and it is positioned immediately adjacent to cell-laden collagen hydrogel 320. This allows for communication between cells and the dentin tubules.

A second microchannel 324 is connected to dentin fragment 318, thus forming the fluid communication between the “outside of the tooth” and the dentin tubules. In such a device, fluid perfusion through the microchannel adjacent to dentin mimics the process of material delivery through the dentin tubules.

As noted previously, the unique anatomical configuration of the pulp-dentin complex imposes constraints on the diffusion of bioactive molecules to the odontoblast layer which—along with the complex 3D organization, constant fluid flow, hydrostatic pressure, and spatiotemporal chemical gradients—cannot be readily replicated in-vitro.

In contrast, FIG. 3B shows an example experimental setup for testing a microfluidic pulp-dentin tissue-chip model. Results from such a test help ascertain efficacy of the engineered material-delivery platform. For example, in pulp-dentin chip microdevice 300, a native dentin fragment is interfaced with a collagen hydrogel embedded with immortalized murine pulp cells (OD21) cells. The entire system is perfusable and transparent for real-time microcopy imaging. Fluid-flow can be controlled with a syringe pump, allowing for interrogation of cell response as a function of chemical gradients, proximity to dentin, and other variables.

To evaluate the effect of dentin thickness on penetration of materials (e.g., dental treatment, biologic materials, nanoparticles, or other materials) and ascertain the maximum thickness at which the materials can be delivered to the odontoblast layer by topical application, the aforementioned pulp-dentin chip may be used to test the transfection efficiency achieved across dentin fragments of increasing thicknesses. Example thicknesses that are comparable are (a) 250 micron, (b) 500 micron, (c) 750 micron, and (d) 1000 micron. For instance, transfection efficiency may be tested with luciferase mRNA and then quantified for RUNX2, such that an upper limit for dentin barrier may be determined to assess whether dentin thickness has no significant effect on the delivery efficiency of mRNA nanoparticles.

Skilled persons will appreciate that the example of FIGS. 3A and 3B also suggests various other combinations of fluidics features are possible and within the scope of this disclosure. For example, a tooth-on-a-chip may include a central channel (e.g., for gel loading), one or more outer channels (e.g., for perfusion), and combinations thereof on one or more sides of a sample. For instance, a single U-shaped channel may be molded to form a single microenvironment on a single side of a dental sample. In another variant, a single U-shaped channel and an elongated gel-loading channel are present on a single side of a dental sample.

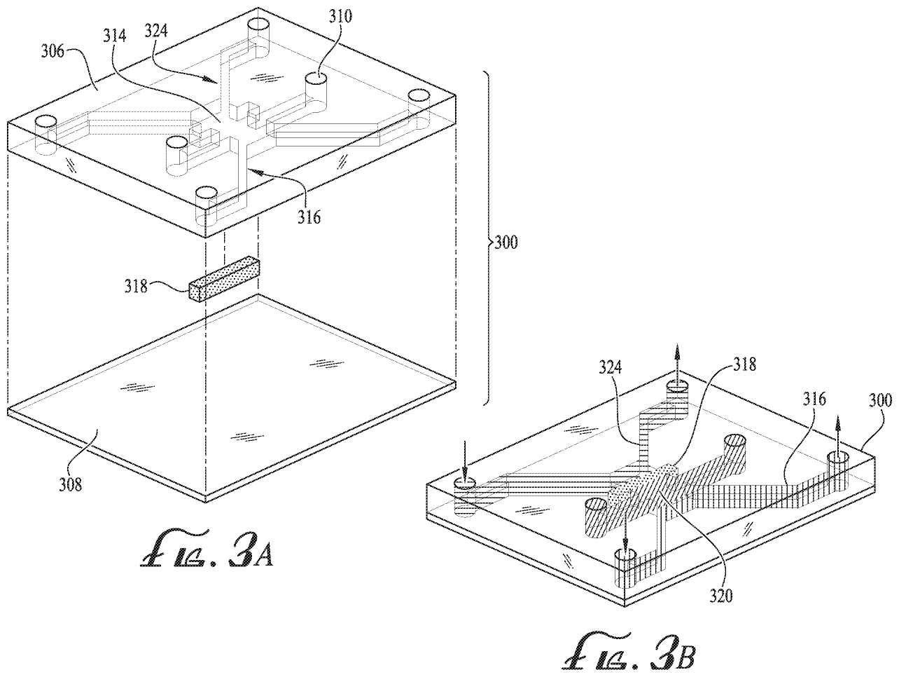

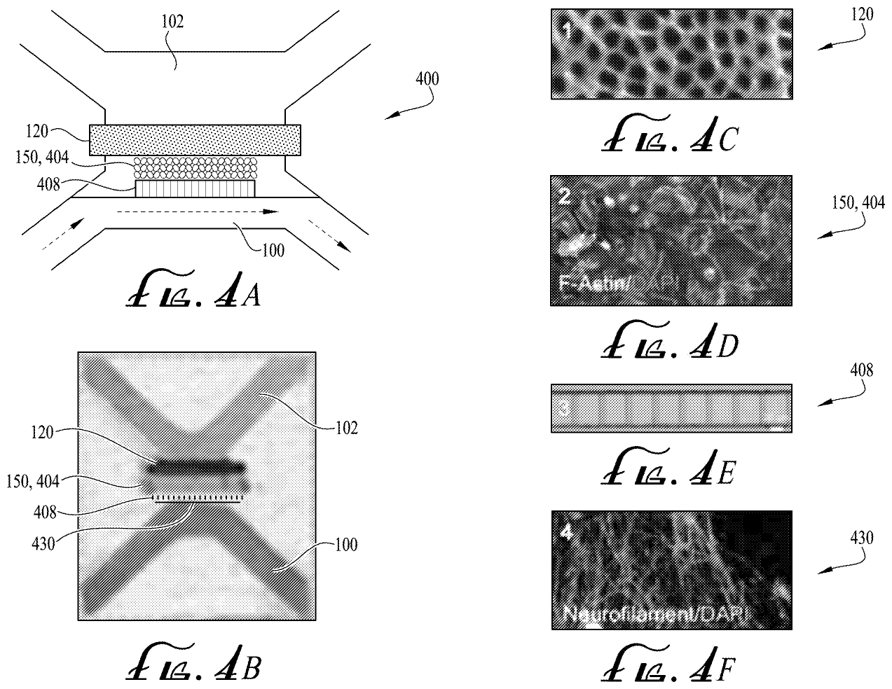

As described previously in connection with FIG. 2B, FIGS. 4A and 4B depict a similar tooth-on-a-chip 400 in which a cell-laden matrix 404 is adjacent dentin sample 120. Unlike the embodiment shown in FIG. 2B, however, cell-laden matrix 150 is separated from direct communication with material flowing through first contained channel 100 by a physical barrier 408 disposed between cell-laden matrix 150 and first contained channel 100. Physical barrier 408 provides a region of controlled flow of materials between first contained channel 100 and cell-laden matrix 150, such as through a series of groves or openings of a desired size.

FIG. 4B shows another variant for an experiment including neurons 430. Components under examination are shown in FIGS. 4C-4F, which provide pictorial views of the following four components: precision-cut dentin fragment 120 in fluid communication with second contained channel 102; a SCAP-laden hydrogel 404 that functions as a pulp-like surrogate in a portion of chamber 108; physical barrier 408 such as seven μm-wide microfabricated grooves through which axonal processes extend towards SCAP-laden hydrogel 404, separately from soma (akin to the native pulp); and freshly isolated neurons 430 seeded away from the pulp-like surrogate (SCAP-laden hydrogel 404) and in fluid communication with first contained channel 100.

The acronym “SCAP” refers to Stem Cells from the Apical Papilla; a unique population of Mesenchymal Stem Cells (“MSCs”) that generally reside in the apical papilla of immature permanent teeth and possess high proliferative potential, including the capacity to give rise to various cell lineages such as osteogenic, odontogenic, neurogenic, adipogenic, chondrogenic, and hepatogenic cells. The term “SCAP-laden” refers to a condition in which a material or volume contains or is filled, loaded, or covered with Stem Cells from the Apical Papilla.

In general, grooves 408 act as an interface between a native dentin fragment (FIG. 4C) and a pulp-like surrogate is formed by loading a controllable hydrogel (gelatin methacryloyl, GeIMA) microenvironment encapsulated with SCAP in a perfusable chamber. This chamber is then interfaced with a microchannel loaded with trigeminal neurons that extend axons towards the SCAP-laden hydrogel via lithography-patterned microgrooves (characterized in subsequent paragraphs) separated from soma—akin to the native pulp where the somal component resides in the trigeminal ganglion. The interface between dentin, pulp-like surrogate, and axons is formed transversally to field of view, and the microdevice is transparent for microcopy imaging. Fluid-flow can be controlled with a syringe pump, allowing for interrogation of cell response as a function of chemical gradients, proximity to dentin, and other variables.

The term “microgroove” refers to one or more furrows, grooves, or channel structures having a width of equal to or less than one millimeter. In some embodiments, a microgroove may comprise one or more furrows, grooves, or channel structures having a width of equal to or less than 750 μm, 600 μm, 500 μm, 400 μm, 300 μm, 250 μm, 200 μm, 150 μm, 100 μm, or 50 μm,

During the pulp regenerative process, the affected pulp has compromised axons that are extirpated during the root canal preparation. Hence, regeneration must rely on the axonal “regrowth” into the pulp chamber. Therefore, the embodiment of 4B closely replicates process of restricted axonal growth towards dentin, while establishing the interface between differentiating pulpal stem cells, native dentin and regenerating neurons, under systematically controllable conditions.

FIG. 4E shows additional details of grooves 408. To fabricate them, a master mold includes two layers of a photoresist (SU-8)—one for the microgrooves and another one for chambers—fabricated on a flat silicon wafer using computer chip lithography techniques. The first photoresist layer is developed to generate micropatterns measuring three μm (H) and 7 μm (W) to give rise to microgrooves upon molding. The second layer is then developed to form fluid-flow channels (200 μm H×1.5 mm W×seven mm L) and both dentin and hydrogel chambers (one mm H×one mm W×three mm L). Upon wafer fabrication, the positive patterns on the wafer are molded with PDMS, by dispensing the PDMS pre-polymer solution onto the silicon substrate and allowing it to set (80° C. for two hours).

The platform allows for fluidic isolation of the two compartments (e.g., corresponding to dental-pulp and dentin) where fluid flow is established from an inlet and an outlet in both dental-pulp and dentin sides. Devices are perfused at a flow rate of 50 μL/h using the medium relevant for the cells in each channel (i.e. odontogenic/neurogenic). Flow rate usage in system will be dependent of final dimensions of system and testing of fabricated devices. Skilled persons will now appreciate that flow rates used in the example devices and designs will vary according to a particular use and the materials and/or cells and biological components utilized. In some embodiments, the flow rate may be from about 0 μL/h (no, or substantially low flow) to about 200 μL/h. In other embodiments, the flow rate may be selected from the group of ranges of 10 μL/h to about 150 μL/h, 20 μL/h to about 120 μL/h, 20 μL/h to about 100 μL/h, 20 μL/h to about 80 μL/h, 30 μL/h to about 70 μL/h, and 40 μL/h to about 60 μL/h.

Cell-laden GeIMA hydrogels are photo-polymerized using a dental curing light, thereby allowing for fine control of physical and mechanical properties of 3D cell microenvironments. Preliminary experiments seeded (2D) and encapsulated (3D) hydrogels with primary trigeminal ganglion neurons and pre-determined the conditions that enhance neurite outgrowth and cell proliferation. Hydrogels of low stiffness (<1.2 kPa) and high porosity (120 μm size, 40% pore ratio) resulted in significantly enhanced neurite outgrowth (FIG. 4F). The GeIMA hydrogels that are used are of low stiffness (GeIMA macromer at 5% (w/v) with a 0.05% (w/v) photoinitiator solution of lithium acylphosphinate (LAP), and photocured with dental light (10 mW/cm²) for five seconds.

Skilled persons will appreciate that many changes may be made to the details of the above-described embodiments without departing from the underlying principles of the invention. The scope of the present invention should, therefore, be determined only by the following claims.

1. A microfluidic device, comprising:
a channel; and
a chamber in fluid communication with the channel and sized to contain a dental sample, the dental sample configured form a semipermeable barrier through which material from the channel is deliverable to the dental sample.

2. The microfluidic device of claim 1, in which the dental sample includes a dentin fragment.

3. The microfluidic device of claim 1, further comprising a portion of the chamber sized to accommodate a cell-laden matrix, the cell-laden matrix being maintained adjacent to the dental sample and in fluid communication with the channel.

4. The microfluidic device of claim 3, further comprising a physical barrier disposed between the cell-laden matrix and the channel, the physical barrier defining a region of controlled flow of the material between the channel and the cell-laden matrix.

5. The microfluidic device of claim 4, in which the physical barrier includes microgrooves.

6. The microfluidic device of claim 1, in which the channel comprises a first channel in fluid communication with a first side of the dental sample, the microfluidic device further comprising a second channel in fluid communication with a second side of the dental sample, the second side being opposite the first side such that the dental sample forms a semipermeable barrier between the first and second channels.

7. The microfluidic device of claim 1, further comprising a biologically inert body in which the channel and chamber are molded.

8. The microfluidic device of claim 7, further comprising a coverslip bonded to a surface of the biologically inert body with at least a portion of the dental sample disposed in the chamber.

9. The microfluidic device of claim 8, in which the biologically inert body comprises a polymer.

10. The microfluidic device of claim 9, in which the polymer is a silicone polymeric material.

11. The microfluidic device of claim 9, in which the polymer comprises polydimethylsiloxane.

12. The microfluidic device of claim 3, in which the cell-laden matrix comprises a hydrogel matrix.

13. The microfluidic device of claim 12, in which the hydrogel matrix is a collagen hydrogel matrix.

14. The microfluidic device of claim 1, further comprising a port disposed at an end of the channel.

15. The microfluidic device of claim 1, in which the port comprises a reservoir.

16. The microfluidic device of claim 1, in which the port comprises a perfusion port.

17. The microfluidic device of claim 1, further comprising a glass coverslip forming a leak-proof seal for the channel and chamber.
